(19) United States
(12) Patent Application Publication
(10) Pub. No.: US 2013/0090566 A1
(43) Pub. Date: Apr. 11, 2013

(54) METHOD AND DEVICE FOR DETECTING A CRITICAL HEMODYNAMIC EVENT OF A PATIENT

(71) Applicants: Jens Mühlsteff, Aachen (DE); Geert Guy Georges Morren, Vissenaken (BE); Christian Meyer, Aachen (DE)

(72) Inventors: Jens Mühlsteff, Aachen (DE); Geert Guy Georges Morren, Vissenaken (BE); Christian Meyer, Aachen (DE)

(73) Assignee: KONINKLIJKE PHILIPS ELECTRONICS N.V., Eindhoven (NL)

(21) Appl. No.: 13/703,704

(22) PCT Filed: Jun. 17, 2011
(86) PCT No.: PCT/IB11/52647

§ 371 Date: Dec. 12, 2012

(30) Foreign Application Priority Data
Jun. 24, 2010 (EP) 10167124.6

(51) Int. Cl. A61B 5/024; A61B 5/0245; A61B 5/0402; A61B 5/00

(52) U.S. Cl. CPC A61B 5/024; A61B 5/7275; A61B 5/742; A61B 5/743; A61B 5/6802; A61B 5/0004; A61B 5/0245; A61B 5/0402; A61B 5/02416
USPC 600/500

(57) ABSTRACT

The invention relates to a method and a device for detecting a critical physiological state of a patient, especially for detecting a critical hemodynamic event. A set of values of physiological parameters is measured, including the heart rate and the pulse arrival time. On the basis of these measurements, a risk assessment is performed including the allocation of a representation of the measured set of values as a vector in a vector space to a risk level representing the risk of the occurrence of a critical hemodynamic event.

### FIELD OF THE INVENTION

The invention relates to the field of detecting a critical physiological state of a patient, especially for detecting an impending critical hemodynamic event. The invention further relates to a corresponding device provided to detect such a critical hemodynamic event.

### BACKGROUND OF THE INVENTION

Patient safety in hospitals has attracted more and more attention in order to prevent medical errors and adverse health care events. There is a clear trend to improve patient safety that calls for better coverage of preventable injuries and death. In this context early detection of critical hemodynamic events, e.g. critical systolic blood pressure drops is still an unmet need in low acuity settings in hospitals as well as in home scenarios. Hemodynamic regulation failures—which can cause serious injuries due to a fall of a fainting patient—are currently not detectable based on state of the art monitoring equipment and existing algorithm approaches. Therefore critical patient states related to such regulation failures are often not or lately noticed by clinical staff in lower acuity settings, since patients are not or seldom monitored. Only basic parameters like heart rate, respiration rate and temperature are acquired, which hardly reflect sudden critical hemodynamic processes. Root causes of regulation instabilities and regulation failures are dehydration, a developing infection, medication incompatibility, wrong drug dosages, etc.

The existing classical sensor portfolio, which was developed primarily for high acuity settings, is not well suited for continuous, reliable and comfortable patient monitoring in low acuity settings in terms of usability, robustness and comfort. For example, blood pressure is measured non invasively by cuff based uncomfortable and bulky systems, only intermittently (often only twice a day or even less). However, a regulation failure can happen in a few seconds.

In general ward state-of-the-art monitoring is still based on the visiting nurse done normally twice a day and is limited to vital signs such as heart rate, respiration rate and temperature. Therefore, critical events or the onset of a patient de-compensation are lately noticed, which can result in suboptimal patient treatment, hospital acquired injuries, longer hospital stays and therefore increased costs.

### SUMMARY OF THE INVENTION

It is an object of the invention to provide a method for detecting a critical hemodynamic event of the patient as described above that allows a permanent analysis of physiological parameters of the patient related to hemodynamic processes and an early detection of impending critical states thereof, so clinical staff, bystanders and/or the patient can be warned early in order to react appropriately. This leads to an improved patient safety in low acuity settings, like emergency waiting rooms, during patient transports, general ward situations, etc. Another object of the invention is to provide a corresponding device for detecting such a critical hemodynamic event.

This object is achieved by a method comprising the features of claim 1, and by a device comprising the features of claim 10.

The method according to the present invention comprises a step of measuring a set of values of physiological parameters, including the heart rate (HR) and the pulse arrival time (PAT). The heart rate is changed by the cardio-vascular regulation system and can be extracted from a measured electrocardiogram (ECG) by state-of-the-art algorithm approaches. The pulse arrival time is sensitive to stroke volume (SV), the pre-ejection period (PEP) and blood pressure changes. It is measured as the time interval between the R-peak in the ECG and a feature of a measured signal related to passing pulse in an artery at a certain body position. This passing pulse can be measured using various modalities such as a photoplethysmogram (PPG) sensor (arterial blood volume change) or a piezo-electric sensor (vibrations or artery dilatation due to passing pulse pressure wave).

The set of physiological parameters being measured in this step can further comprise:

the pulse transit time (PTT), estimated as time interval from the closure of the aortic valve until the onset of an arriving pulse;

left ventricular ejection time (LVET), which can be estimated from PPG pulse contour analysis signals or by analysis of heart sounds;
pre-ejection period (PEP), measured as part of the PAT or by analysis of heart sounds;

pulse shape features such as the occurrence and morphology of a dicrotic notch in a PPG;

the quantified activity level, derived from accelerometer signals;

the posture of the patient, derived e.g. from an acceleration sensor.

The list given above only contains examples of body parameters to be measured and is not meant to be delimiting the set of measured values of physiological parameters in the sense of the present invention.

On the basis of the measured set of values of physiological parameters, a step of performing a risk assessment for estimating the probability of the occurrence of a critical hemodynamic event is performed.

In this risk assessment, a representation of the measured set of values as a vector \vec{R} in a vector space is allocated to a predetermined risk level that represents the risk of the occurrence of a critical hemodynamic event.

For example, the measured values of the heart rate and the pulse arrival time at one point of time t can be represented by the vector \vec{R}_{R} (t)=[HR(t), PAT(t)] in a two-dimensional vector space, a first dimension thereof representing the parameter of the heart rate and the second dimension representing the pulse arrival time. This vector \vec{R}_{R} can be allocated to a predetermined region of this two dimensional vector space that represents a certain risk level. For example, if the vector \vec{R}_{R} points into a region of the vector space that represents a high risk of the occurrence of a critical hemodynamic event, a corresponding warning can be displayed. A display of the vector as such, the corresponding present measured values, etc., can represent a further visualization of the occurrence of the critical event.

This kind of risk assessment is based on a finding that certain combinations of different values of physiological parameters represent a certain risk for the occurrence of critical physiological states. This stands especially for the heart rate and the pulse arrival time. For example, the present inventors have found that an increase of the heart rate combined with an increase of the pulse arrival time refers to an impending critical state, while a PAT decrease together with an HR increase may not necessarily be critical. With the present method, however, a critical combination of both HR and PAT is detected and analyzed automatically.

According to a preferred embodiment of the present invention, the risk level is represented by a predetermined region of the vector space.

According to another preferred embodiment, said vector space comprises at least two dimensions, namely a first dimension representing the heart rate and a second dimension representing the pulse arrival time. Preferably, the origin of said vector space is a reference point defined by a set of values (HR₀, PAT₀) of the heart rate and the pulse arrival time measured at a point of time t₀ or determined as average values from a pre-defined time interval [t₀−ΔT . . . t₀] e.g. extracted before the monitoring period starts at t₀.

In this preferred embodiment, a basal state of the patient is defined by the reference point. The values HR₀ and PAT₀ defining this reference point are the measured values at the time t₀ or determined as average values from a pre-defined time interval [t₀−ΔT . . . t₀] e.g. extracted before the monitoring period starts at t₀. The following measurements of sets of values of the physiological parameters are assessed in relation to this vector space.

Preferably the predetermined region representing a risk level is delimited in the second dimension by a minimum threshold value PAT_{Thres} for the pulse arrival time.

This means that in the case in which the PAT falls below PAT_{Thres}, the occurrence of a critical hemodynamic state can be concluded, independent from the heart rate.

According to another preferred embodiment, for values of the heart rate lower than HR₀, the predetermined region is further delimited by a threshold formed by a slope ascending to higher values of the pulse arrival time with decreasing values of the heart rate.

For example, if the end point of the vector \vec{R}(t)=[HR(t)/HR₀; PAT(t)/PAT₀] lies higher than the slope beginning at HR=HR₀ and ascending with HR values running in the negative direction from HR₀, a critical combination of HR and PAT can be detected.

According to another preferred embodiment, the risk assessment further includes a trend analysis, comprising the determination of the direction and/or the length of a vector \vec{R}(t)−\vec{R}_{ref}, wherein \vec{R}(t) represents the measured set of values, and \vec{R}_{ref} denotes a time dependent adaptive reference point, wherein \vec{R}_{ref} is changed in case of a significant variation of \vec{R}(t) within a predetermined time interval.

The trend analysis takes into account that a reference point \vec{R}_{ref} may change with time. For example, \vec{R}_{ref} is used as long as the direction of the vector \vec{R}(t)−\vec{R}_{ref} compared with a short term variation of \vec{R}(t)−\vec{R}(t−Δt) does not change significantly. In this context Δt is a parameter to be defined appropriately. The “significance” of such a change can be defined by the following threshold:

$$\frac{\vec{R} ( t ) - {\vec{R}}_{ref}}{\vert \vec{R} ( t ) - {\vec{R}}_{ref} \vert} \cdot \frac{\vec{R} ( t ) - \vec{R} ( t - Δ t )}{\vert \vec{R} ( t ) - \vec{R} ( t - Δ t ) \vert} < Th$$

If this threshold Th is crossed, a new reference point \vec{R}_{ref} is determined. The vector \vec{R}(t)−\vec{R}_{f} shows a development of the physiological state of a patient, possibly indicating a pathological trend. This vector \vec{R}(t)−\vec{R}_{ref} can also be shown graphically to visualize this trend.

Preferably, the method according to the present invention includes a visualization step of displaying the vector \vec{R}(t) within the vector space and/or the measured set of values on a screen.

More preferably, this visualization step includes graphically displaying the vector \vec{R}(t)−\vec{R}_{ref} on a screen.

The visualization step can preferably include graphically displaying the present risk level on a screen.

A device according to the present invention for detecting a critical hemodynamic event of a patient, especially an impending critical hemodynamic event, comprises sensors for measuring a set of values of physiological parameters, said physiological parameters including the heart rate and the pulse arrival time, and a calculating device for processing the measured values, said calculating device being provided to perform a risk assessment including the allocation of a representation of the measured set of values as a vector \vec{R}(t) in a vector space to a risk level representing the risk of the occurrence of a critical hemodynamic event.

Preferably said sensors are provided to perform a reference measurement in which a set of values of the heart rate and the pulse arrival time is measured at a point of time T₀, or determined as average values from a pre-defined time interval [T₀−ΔT . . . T₀] e.g. extracted before the monitoring period starts at T₀, said set of values defining a reference point. Preferably said calculating device is provided to allocate a representation of the measured set of values as a vector \vec{R}(t) to a predetermined region of a two dimensional vector space comprising a first dimension representing the heart rate and a second dimension representing the pulse arrival time, the origin of this vector space being said reference point.

According to a preferred embodiment, said calculating device is provided to determine the direction and/or the length of a vector \vec{R}(t)−\vec{R}_{f}, wherein \vec{R}(t) represents a measured set of values, and \vec{R}_{ref} denotes a time dependent adaptive reference point, said calculating device being further provided to change \vec{R}_{ref} in case of a significant variation \vec{R}(t) within a predetermined time interval.

According to another preferred embodiment, said device further comprises a display for displaying at least of the following: a measured set of values, a vector \vec{R}(t) within the vector space, the vector \vec{R}(t)−\vec{R}_{ref}, the present risk level.

According to another preferred embodiment, said sensors are integrated into a body worn system that is wirelessly connected to a monitoring station comprising said calculating device.

### BRIEF DESCRIPTION OF THE DRAWINGS

These and other aspects of the invention will be apparent from and elucidated with reference to the embodiments described hereinafter.

In the drawings:

FIG. 1 represents a diagram showing graphically the allocation of a representation of a measured set of two values as a vector in a two-dimensional vector space to a risk level;

FIG. 2 is a view of a screen shot representing a visualization of the risk assessment according to the present invention; and

FIG. 3 is a schematic view of one embodiment of a device according to the present invention.

### DETAILED DESCRIPTION OF EMBODIMENTS

In the following a first embodiment of a method for detecting a critical hemodynamic event of a patient is described. In this method a set of values of physiological parameters of the patient is measured permanently to acquire sets of values related to different points of time t. The values represent the output of a plurality of sensors, including one sensor for measuring a value of the heart rate (HR) and another sensor for determining a value of the pulse arrival time (PAT). PAT can be measured as the time interval between the R-peak in the ECG and a feature of a measured signal related to a passing pulse in an artery at a certain body position using, for example, a PPG sensor or a piezoelectric sensor. For each point of time t, a set of two values is gained, namely one value for the heart rate (HR) and one for the pulse arrival time (PAT). As it will be laid out in the following, this combination of two physiological parameters can be used to derive a certain risk of the occurrence of an impending critical hemodynamic event.

It is noted that this embodiment of the present invention is not limited to measuring only the heart rate (HR) and the pulse arrival time (PAT) but can be extended to measure additional physiological parameters and take them into account, for example, the pulse transit time (PTT), the left ventricular ejection time (LVET), the pre-ejection period (PEP), etc. Additional information for a risk assessment can include also detected arrhythmias based on the ECG by state-of-the-art algorithms, that are for example used in cardiographs, as well as posture information and/or the physical activity level of the subject.

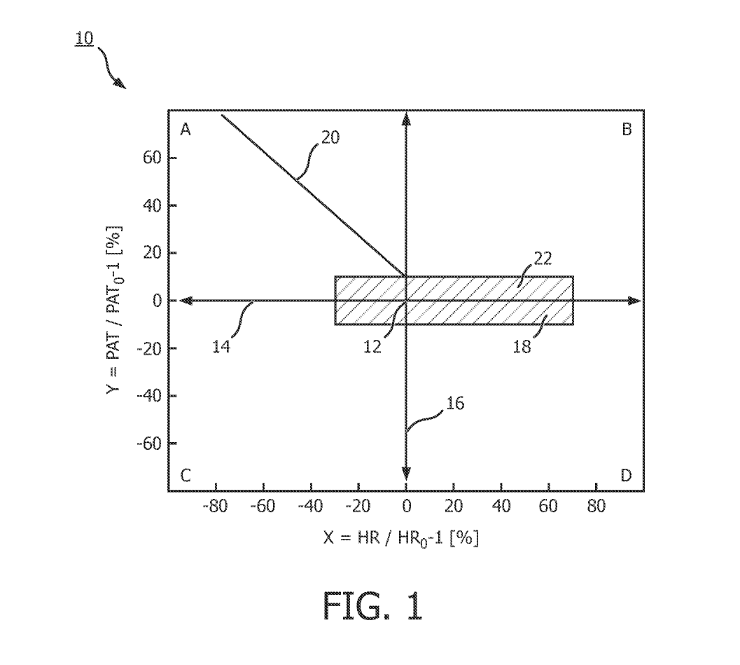

A set of two values of physiological parameters measured at a certain time t can be represented as a vector \vec{R}_{R}(t) in a two-dimensional vector space 10, as it is represented by the Euclidian plane in FIG. 1, comprising two dimensions. The first dimension (corresponding to the horizontal axis 14 of this coordinate system) represents the heart rate (HR) while the second dimension (represented by the vertical axis 16 in FIG. 1) represents the pulse arrival time (PAT). One point in this plane represents a set of values relating a pulse arrival time to a heart rate.

This coordinate system also represents a vector space 10 wherein a set of two values can be represented as a vector \vec{R}(t). The two components of this vector \vec{R} represent the two values of the measured physiological parameters. Because these parameters change, direction and length of vector \vec{R} may change with time.

The origin 12 of this vector space 10 is a reference point defined by a set of two values (HR₀, PAT₀) of the heart rate (HR) and pulse arrival time (PAT) measured at a point of time t₀. It is also possible to define this reference point by taking an average of the measured values for HR and PAT over a certain basal period of time and to calculate HR₀, and PAT₀ as the average of these values.

To define a relation between this basal reference point (HR₀, PAT₀) and vectors \vec{R}(t) defined by following measurements of sets of values for HR and PAT, \vec{R} can be represented as:

$$\vec{R} ( t ) = [ \begin{matrix}\frac{HR ( t )}{{HR}_{0}} \\ \frac{PAT ( t )}{{PAT}_{0}}\end{matrix} ]$$

Within the vector space 10, predetermined regions represent risk levels to determine the occurrence of an impending critical hemodynamic event as, for example, a syncope. The hatched rectangular area 18 around the basal reference point 12 denotes a normal physiological range for the pulse arrival time and the heart rate. Out of this normal physiological range 18, different risk regions are defined.

In the upper right quadrant B in vector space 10, with a value PAT_{Thres} as a minimum threshold value 22 for the pulse arrival time and a heart rate higher than HR₀, the combined increase of PAT and HR represents a critical hemodynamic status. If the present vector \vec{R} points into this region, it is allocated to an increased risk level representing an increased risk of the occurrence of a critical hemodynamic event such as an impending syncope. This region is extended toward the upper left quadrant A but delimited in the downward direction by a slope 20 beginning at the vertical coordinate 16 with HR₀ and PAT=PAT_{Thres} and running from this origin in the upper left direction, i.e. ascending to higher values of the pulse arrival time with decreasing values of the heart rate. If \vec{R} points in the area of the upper left quadrant A that is above this threshold defined by the slope 20, the hemodynamic state of the patient is also critical. Above the slope 20, the combined HR decrease and PAT increase is then critical. However, below this slope 20, with the same value for the heart rate HR, a low measured PAT does not represent a risk, i.e. the vector \vec{R} points to a region with a decreased risk level that is not critical.

The above can be summarized in that after a step of measuring a set of values of physiological parameters including the heart rate and the pulse arrival time, the step of a risk assessment is performed in which a representation of the measured set of values as a vector \vec{R} in a vector space 10 is performed, and this representation \vec{R} is allocated to a risk level in this vector space, represented by a predetermined region of the vector space. In the example given above, the predetermined region of increased risk level is delimited to the downward direction in the upper right quadrant B by the threshold value PAT_{Thres} for the pulse arrival time, and by the slope 20 in the upper left quadrant A.

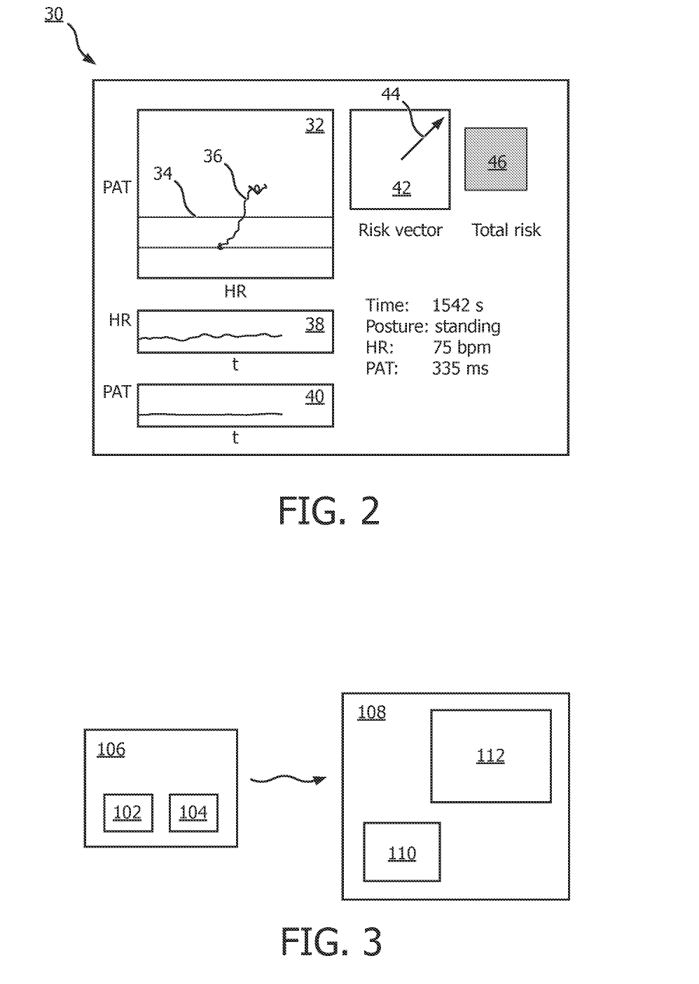

The screenshot in FIG. 2 represents a visualization of the measured values of the physiological parameters, together with a part of the two-dimensional vector space 10 in a window 24. The vectors \vec{R} as such are not shown but the chronological progression 36 of the end points of these vectors \vec{R}, representing sets of values (HR, PAT). PAT_{Thres} is also marked by a horizontal line 34 in this window 32. Each point in the line 36 in the window 32 showing the chronological development of the combination of HR and PAT represents one set of values (HR,PAT) at a certain point of time t. The values for HR and PAT as such are also displayed in separate windows 38 and 40, respectively.

On the right side of the screenshot 30, another rectangular window 42 is shown with a representation of a vector 44, pointing from an origin in the middle of the window 42 outwardly. This vector 44 is a vector \vec{R}(t)−\vec{R}_{f} that shows a trend of the physiological status of the patient.

While the vector \vec{R} represents one present set of values of the heart rate HR and the pulse arrival time PAT, as described above, the vector \vec{R}_{ref} represents an adaptive reference point at a time t_{ref}. That is, \vec{R}(t)−\vec{R}_{f} represents a development from the time t_{ref} to a present time t when the measurement was taken that is represented by \vec{R}. The reference point \vec{R}_{ref} is maintained as long as the direction of the vector \vec{R}(t) \vec{R}_{f} compared with a short time variation of \vec{R}(t)−\vec{R}(t−Δt) does not change significantly (Δt is a parameter designating a time period to be defined appropriately). The “significance” of such a change is defined by a threshold value Th as follows:

$$\frac{\vec{R} ( t ) - {\vec{R}}_{ref}}{\vert \vec{R} ( t ) - {\vec{R}}_{ref} \vert} \cdot \frac{\vec{R} ( t ) - \vec{R} ( t - Δ t )}{\vert \vec{R} ( t ) - \vec{R} ( t - Δ t ) \vert} < Th$$

If the threshold Th is exceeded, this denotes a significant change of the short term variation. In this case the reference point \vec{R}_{ref} is adapted, i.e. a new adaptive reference point \vec{R}_{ref} is used at the time point t. For the evaluation of the risk the value of:

$$\frac{\vec{R} ( t ) - {\vec{R}}_{ref}}{\vert \vec{R} ( t ) - {\vec{R}}_{ref} \vert} \cdot {\vec{e}}_{x}$$

and the length:

|\vec{R}(t)−\vec{R}_{ref}|

are taken into account, representing the direction of a change of \vec{R} here corresponding to the coordinate x and the extent of the change.

From the graphic visualization by the vector 44 in window 42 it is possible to draw conclusions on the development of a physiological state of the patient.

There is another window 46 right to the window 42, being a color window 46 showing a color that represents the risk level. This total risk level is the result of the risk assessment as described before, taking into account the vector \vec{R} being allocated to a region of the vector space 10, and the trend towards a critical physiological state represented by \vec{R}−\vec{R}_{ref}. For example, this window 46 can show a red warning color when there is a critical state, while it shows a yellow color when there is a trend towards a critical state. The color designation can be chosen suitably in the graphic visualization represented by the screenshot 30. It is also possible to provide the vector 44 in window 42 with a respective color designation.

It is, of course, possible to show other features in the graphic visualization, for example, a context information about the patient's posture, an information about the time development of the physiological state, detected arrhythmias, etc.

The device for detecting a critical hemodynamic event of a patient may comprise corresponding sensors for measuring a set of physiological parameters that are to be measured and that will be taken into account in the risk assessment step to judge the risk of the occurrence of a critical hemodynamic event. A suitable calculating device may be provided for processing the measured values, and these values can be displayed in an x-y-plot, as represented by the vector space 10 in FIG. 1, as well as the vector \vec{R}, the vector \vec{R}−\vec{R}_{ref}, the present risk level and so on. For this display a screen of a monitor may be provided. Such a device is suitable to be used in a lower acuity setting like an emergency waiting room, in a patient transport vehicle, in a general ward situation at home, or at any other place as desired.

One example for such a device 100 is shown in FIG. 3. In this embodiment the device 100 comprises sensors 102, 104 integrated into a body worn system 106 that is wirelessly connected to a monitoring station 108. The physiological parameters measured by the sensors 102, 104 are transmitted to the monitoring station 108 to be received and processed by a calculating device 110 integrated into the monitoring station 108. The monitoring station 108 also comprises a display 112 for displaying the results of the processing according to the screenshot 30 in FIG. 2. Although this is not shown in this embodiment, the monitoring station 108 may further comprise a device for transmitting a warning signal to a central monitoring unit in an architecture with plural monitoring stations 108 communicating with this unit.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A method for risk assessment of a critical hemodynamic event of a patient, comprising the steps of:
measuring a set of values of physiological parameters, said physiological parameters including heart rate (HR) and pulse arrival time (PAT), wherein said set of values includes a heart rate (HR) value and a pulse arrival time (PAT) value, and
performing a risk assessment by a calculating device, said risk assessment including the allocation of a representation of the measured set of values as a two-dimensional vector \vec{R}(t) in a two-dimensional vector space to a risk level representing the risk of the occurrence of a critical hemodynamic event.
Wherein a first dimension of the two-dimensional vector represents the heart rate (HR) value and a second dimension of the two-dimensional vector represents the pulse arrival time (PAT) value,
Wherein the allocation is based on a combination of the heart rate (HR) value and the pulse arrival time (PAT) value in the vector, which combination represents the risk, and
Displaying the vector \vec{R}(t) within the vector space.

2. The method according to claim 1, wherein said risk level is represented by a predetermined region of said vector space.

3. (canceled)

4. The method according to claim 1, wherein the origin of said vector space is a reference point defined by a set of reference values (HR₀, PAT₀) of the heart rate (HR) and the pulse arrival time (PAT)
measured at a point of time t₀ or
calculated as an average of heart rate (HR) values and pulse arrival time (PAT) values measured over a certain basal period of time respectively.

5. The method according to claim 2, wherein said predetermined region is delimited in the second dimension by a minimum threshold value PAT_{Thres} for the pulse arrival time (PAT).

6. The method according to claim 4, wherein for values of the heart rate (HR) lower than HR₀, said predetermined region is further delimited by a threshold formed by a slope ascending to higher values of the pulse arrival time (PAT) with decreasing values of the heart rate (HR).

7. The method according to claim 4, wherein said risk assessment includes a trend analysis, comprising the determination of the direction and/or the length of a vector \vec{R}(t)−\vec{R}_{ref}, wherein \vec{R}(t) represents the measured set of values, and \vec{R}_{ref} denotes a time dependent adaptive reference point, wherein \vec{R}_{ref} is changed in case of a significant variation of \vec{R}(t) within a predetermined time interval.

8. The method according to claim 4, wherein displaying the vector \vec{R}(t) within the vector space further includes displaying the measured set of values on a screen.

9. The method according to claim 7, wherein said visualization step further includes graphically displaying the vector \vec{R}(t)−\vec{R}_{ref} on a screen.

10. A device for risk assessment of a critical hemodynamic event of a patient, comprising:
sensors for measuring a set of values of physiological parameters, said physiological parameters including heart rate (HR) and pulse arrival time (PAT), wherein said set of values includes a heart rate (HR) value and a pulse arrival time (PAT) value and
a calculating device for processing the measured values, said calculating device being provided to performing a risk assessment including the allocation of a representation of the measured set of values as a two-dimensional vector \vec{R}(t) in a two-dimensional vector space to a risk level representing the risk of the occurrence of a critical hemodynamic event
Wherein a first dimension of the two-dimensional vector represents the heart rate (HR) value and a second dimension of the two-dimensional vector represents the pulse arrival time (PAT) value,
Wherein the allocation is based on a combination of the heart rate (HR) value and the pulse arrival time (PAT) value in the vector, which combination represents the risk:
A display adapted to display the vector \vec{R}(t) within the vector space.

11. The device according to claim 9, wherein said sensors are provided to perform a reference measurement in which
a set of reference values (HR₀, PAT₀) of the heart rate (HR) and the pulse arrival time (PAT) defining a reference point is measured at a point of time (t₀) or
heart rate (HR) values and pulse arrival time (PAT) values are measured over a certain basal period of time, and the set of reference values (HR₀, PAT₀) is calculated as an average of the measured values of the heart rate HR values and the pulse arrival time (PAT) values, respectively.

12. The device according to claim 9, wherein an origin of the vector space is the reference point.

13. The device according to claim 9, wherein said calculating device is provided to determine the direction and/or the length of a vector \vec{R}(t)−\vec{R}_{ref}, wherein R represents the measured set of values, and \vec{R}_{ref} denotes a time dependent adaptive reference point, and to change \vec{R}_{ref} in case of a significant variation of \vec{R}(t) within a predetermined time interval.

14. The device according to one of claims 9, further comprising a display for displaying at least one of the following:
the measured set of values;
the vector \vec{R}(t)−\vec{R}_{ref};
the present risk level.

15. The device according to claim 9, wherein said sensors are integrated into a body worn system that is wirelessly connected to a monitoring station comprising said calculating device.
